(19) United States
(12) Patent Application Publication
(10) Pub. No.: US 2007/0265634 A1
(43) Pub. Date: Nov. 15, 2007

(54) OSTEOTOMY GUIDE

(75) Inventor: ROBERT WEINSTEIN, ATLANTA, GA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: ERMI CORPORATION, DECATUR, GA (US)

(21) Appl. No.: 11/694,652

(22) Filed: Mar. 30, 2007

Related U.S. Application Data
(60) Provisional application No. 60/787,845, filed on Mar. 31, 2006.

(51) Int. Cl. A61B 17/15

(52) U.S. Cl. 606/87

(57) ABSTRACT

The present invention provides improved systems and methods for facilitating cuts in an item. In one embodiment, a surgical apparatus is provided that includes a first saw guide having a first base portion with a first bore and a first guide portion comprising a pair of substantially parallel walls spaced apart to guide the surgical saw; a second saw guide pivotably connected to the first saw guide, wherein the second saw guide includes a second base portion with a second bore and a second guide portion comprising a pair of substantially parallel walls spaced apart to guide the surgical saw; and a locking mechanism configured to selectively discourage relative movement between the first saw guide and the second saw guide.

### CROSS-REFERENCE SECTION

This application claims the full benefit and priority of U.S. Provisional Application No. 60/787,845 entitled Osteotomy Guide, filed Mar. 31, 2006, which is incorporated herein in its entirety.

### BACKGROUND OF THE INVENTION

Angular and linear osteotomy procedures are commonly performed in orthopedic surgery. An osteotomy is a surgical operation whereby a bone is cut to shorten, lengthen, or change its alignment in order to correct an angular deformity. Linear osteotomies which entail making one or more cuts substantially perpendicular to the axis of the bone are often performed to shorten or lengthen a bone. When lengthening is desired, the osteotomy procedure is performed to allow interposition of graft material.

Angular deformity of a bone is another malady that may be addressed using osteotomy procedure. FIG. 1 illustrates a hallux valgus condition which is an example of an angular deformity. To correct this condition, a wedge of material may be removed from the bone such that when the cut ends of the bone are reapproximated the angulation deformity in the bone is reduced or eliminated as generally shown in FIG. 2.

The success of an osteotomy procedure is often dependent on the precision of the cuts made by the surgeon. Precise cuts allow substantially flush seating of bone graft material in the case of the linear osteotomy. For accurate reapproximation of the bone ends in the angular osteotomy procedures, the cuts should be in the same plane and converge on a common apex. When there is a significant discrepancy between the planes of the cuts in either procedure, there is significant risk for delayed or failure of bone healing, in addition to difficulty in fixation when surgical implants are used to repair these osteotomies.

In some prior art methods, the surgeon “free hands” the cuts thereby creating a potential for significant misalignment of the cut surfaces when the bone ends are reapproximated. As a result, some have suggested using cutting guides to improve the precision of the cuts. Examples of these guides may be seen in U.S. Pat. No. 4,627,425 and U.S. Pat. No. 4,750,481 to Reese, U.S. Pat. No. 4,335,715 to Kirkley and U.S. Pat. No. 4,349,018 to Chambers. These devices require the “free handing” of at least one bone cut in angular osteotomies and are cumbersome to use.

Accordingly, what is needed are apparatus and methods to provide a cutting guide for use in osteotomy procedures that address deficiencies in the prior art some of which are discussed above.

### BRIEF SUMMARY OF THE INVENTION

The present invention provides improved systems and methods for guiding a saw. More particularly, embodiments of the present invention provide one or more saw guides for facilitating a linear cut or a wedge shaped cut in an item which address deficiencies in the art, some of which are discussed above.

In one embodiment, an apparatus for guiding a surgical saw during osteotomy procedures is provided. This apparatus includes a first saw guide having a first base portion with a first bore and a first guide portion comprising a pair of substantially parallel walls spaced apart to guide the surgical saw; a second saw guide pivotably connected to the first saw guide, wherein the second saw guide includes a second base portion with a second bore and a second guide portion comprising a pair of substantially parallel walls spaced apart to guide the surgical saw; and a locking mechanism configured to selectively discourage relative movement between the first saw guide and the second saw guide.

In a further embodiment, a method of aligning a surgical saw to make complementary cuts on a bone is provided. This method includes the steps of: securing a guide wire to a bone; threading a first saw guide onto the guide wire wherein the first saw guide includes a pair of substantially parallel walls spaced apart to guide the saw; threading a second saw guide onto the guide wire wherein the second saw guide includes a pair of substantially parallel walls spaced apart to guide the saw; and selectively engaging a locking mechanism to discourage relative movement of the first saw guide relative to the guide wire.

In another embodiment, an apparatus for facilitating a linear cut in a bone is provided. This apparatus includes: a saw guide having a base portion with a bore extending there through and a guide portion comprising a pair of substantially parallel walls spaced apart to guide a cutting apparatus; a spacer having a second bore and a cylindrical section having substantially the same central axis as the second bore and sized to be received in the bore of the saw guide; a locking mechanism configured to discourage relative movement between the saw guide and the spacer; and a guide wire having a first end configured to engage a bone and sized to cooperate with the second bore.

In a further embodiment, an apparatus for guiding a bone cutting apparatus is provided. This apparatus includes: a first saw guide comprising (1) a first base portion forming a first bore and a cylindrical section having substantially the same central axis as the first bore and (2) a first guide portion comprising a pair of substantially parallel walls spaced apart to accept the cutting apparatus wherein the parallel walls of the first guide portion are substantially parallel with a longitudinal axis of the first bore; a second saw guide comprising (1) a second base portion forming a second bore configured to receive the cylindrical section and forming a threaded hole intersecting with the second bore, and (2) a second guide portion comprising a pair of substantially parallel walls spaced apart to accept the cutting apparatus there between wherein the parallel walls of the second guide portion are substantially parallel with a longitudinal axis of the second bore; and a locking mechanism comprising a set screw sized to cooperate with the threaded hole such that the set screw can be selectively tightened against the cylindrical section of the first saw guide to discourage relative movement of the first guide relative to the second guide.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 a schematic diagram that illustrates a hallux valgus condition and a wedge cut for correcting the condition.

FIG. 2. is a schematic drawing that illustrates the same skeletal structure as FIG. 1 but after the wedge has been removed and the ends of the bones reapproximated.

FIG. 3 is a schematic drawing of osteotomy guide in accordance with an embodiment of the present invention.

FIG. 4 is an exploded view of the apparatus 10 in accordance with an embodiment of the present invention.

FIG. 5 is a top view of apparatus 10 illustrating relative rotational movement first guide the second guide in accordance with an embodiment of the present invention.

FIG. 6 is a schematic drawing of an alternative first guide in accordance with an embodiment of the present invention.

FIG. 7 is a schematic drawing of an alternative first guide in accordance with an embodiment of the present invention.

FIG. 8. is a schematic drawing of an embodiment of the present invention for linear osteotomy procedures.

FIG. 9 is a schematic drawing illustrating cuts made using an embodiment of the present invention.

FIGS. 10-16 are schematic drawings showing some of the osteotomy procedures that may be facilitated using embodiments of the present invention.

### DETAILED DESCRIPTION OF THE INVENTION

The present inventions will now be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

### GENERAL OPERATION AND STRUCTURE

The present invention generally relates to cutting guides for use in osteotomy procedures. Embodiments of the present invention provide a cutting guide that is secured to a bone in any known or develop manner and restricts movement of a surgical cutting saw to improve bone cutting precision.

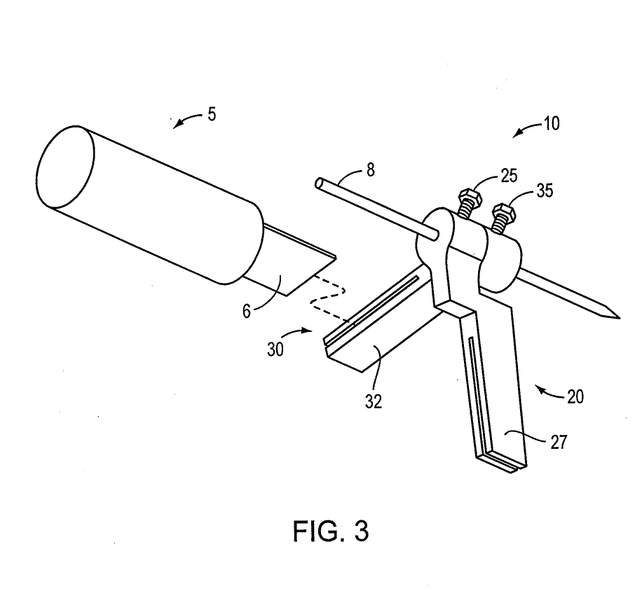

FIG. 3 illustrates a surgical saw 5, a guide wire 8 and apparatus 10 that facilitate making two cooperating cuts in a bone in accordance with an embodiment of the present invention. This apparatus could be used to facilitate wedge cuts in a bone to correct angular deformities. The guide wire 8 is secured to the bone and the apparatus 10 threaded onto the guide wire 8 and locked into place using a set screw 25. The blade 6 of the surgical saw 5 is inserted into saw guides 20 and 30 to make the bone cuts. Of course, the surgical saw may be inserted into the saw guides in any sequence.

Apparatus 10 includes a first guide 20 and a second guide 30, each having a saw guide portion 27, 32 respectively. The first and second guides 20, 30 fit together and rotate about a common axis to facilitate two cuts to the bone having planes that intersection proximate the axis of rotation. As one of ordinary skill in the art will appreciate, however, the two guides may alternatively be configured to facilitate cuts having an intersection point other than the axis of rotation. For example, the saw guide portions 27, 32 of the first or second guide portions 20, may be oriented in planes that do not intersect proximate the axis of rotation.

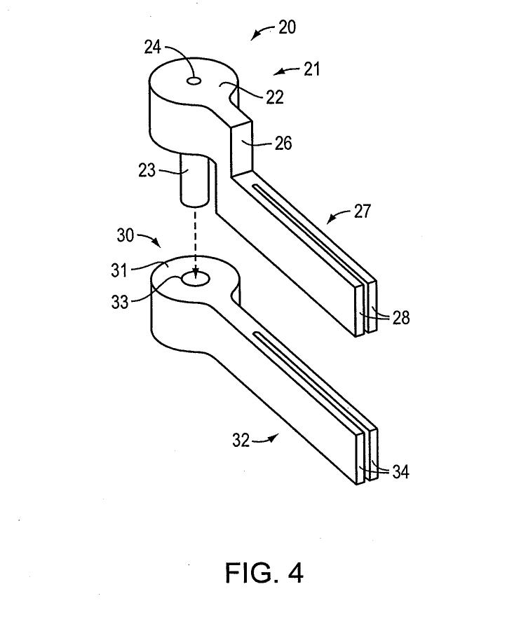

FIG. 4 illustrates the first guide 20 and second guide 30 of apparatus 10. The first guide 20 includes a base portion 21 and a saw guide portion 26. The base portion 21 generally comprises a first cylindrical section 22 and a second cylindrical section 23 where the centers of the two cylindrical sections are proximate the same axis. The first cylindrical section 22 has a generally larger diameter than the second cylinder section 23. A bore 24 is formed proximate the centers of the two cylindrical sections and extends through the first and second cylindrical sections 22, 23.

The first cylindrical section 22 also includes a threaded hole (not shown) formed in the outer surface of the cylindrical section 22 and extending to the bore 24. The threaded hole is oriented substantially perpendicular with the substantially smooth bore 24. A locking set screw 25 is configured to be threaded into the threaded hole and in use, is tightened against the guide wire 8 thread through the bore 24 to discourage movement of the assembly 10 (e.g., rotation and pistoning movement).

Extending from the first cylindrical section 22 is a saw guide support 26, which provides a connection between the saw guide portion 27 and the first cylindrical section 22. As shown in FIG. 4, the saw guide support 26 positions the saw guide portion 27 at a location space apart from the second cylindrical section 23 where the upper surface of the saw guide portion 27 is within substantially the same plane as the bottom surface of the first cylindrical section 22.

The saw guide portion 27 includes two substantially parallel wall portions 28 that are space apart to accept a surgical saw therebetween. These wall portions 28 are substantially parallel with a plane passing through the central axis of the bore 24.

The second guide 30 also includes a base portion 31 and a saw guide portion 32. The base portion 31 is cylindrical in shape with a through bore 33 formed therein. The bore 33 is sized to accept the second cylindrical section 23 of the first guide 20.

As with the first guide 20, the second guide also includes a saw guide portion 32 that includes two substantially parallel walls 34. These walls are space apart to accept a surgical saw therebetween. The walls extend from the base portion 31 and are substantially parallel with a plane that passes through the center axis of the bore 33.

To assemble the apparatus 10, the second cylindrical section 23 of the first guide 20 is positioned into the bore 33 of the second guide 30 such that the second guide 30 may rotate about the second cylindrical section 22 of the first guide 20. As assembled, the lower surfaces of saw guide portion 27 and saw guide 32 are within substantially the same plane.

In the embodiment shown in FIGS. 3-4, the base 31 of the second guide 30 includes a threaded hole (not shown) extending from the exterior surface of the base 31 to the bore 33. This threaded hole is engaged by a set screw 35 that may be tightened against the second cylindrical section 23 of the first guide 20 such that relative angular motion of the first and second guides 20, 30 is discouraged.

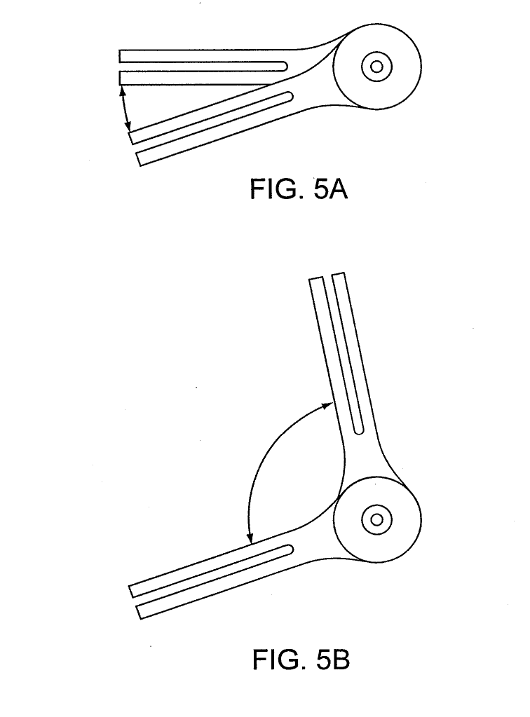

As shown in FIGS. 5a, 5b, various angles may be achieved between the two saw guide portions. In the illustrated embodiment, the angle between the saw guide portions of the first and second guides may secured at any desired angle between 10 and 350 degrees.

### METHODS OF USE

As discussed, embodiments of the present invention provide guides for improved precision of osteotomy procedures. The following paragraphs will describe a method for correcting a hallux valgus condition in accordance with an embodiment of the present invention. It should be understood, however, that embodiments of the present invention may be used in connection with any osteotomy procedures.

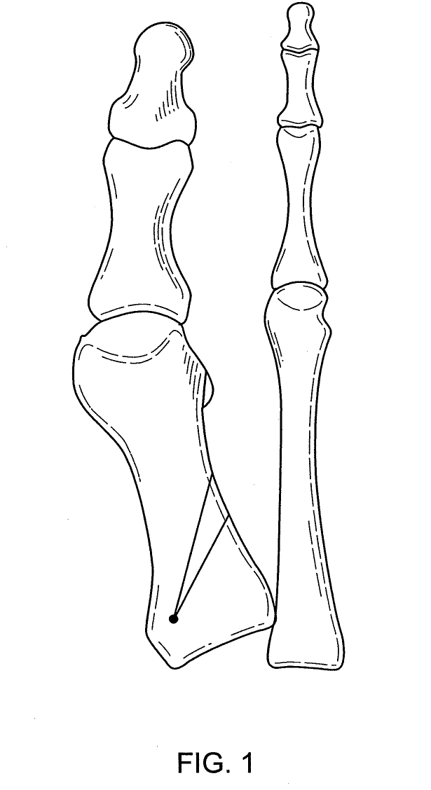

A hallux valgus is a deformity of the big toe where the joint at the base of the toe projects outward as generally shown in FIG. 1. To correct this problem, osteotomy procedures are performed to remove a wedge shaped portion of the bone as generally illustrated.

An initial step is to attach guide wire 8 to the bone using well known or developed attachment methods at a location proximate the apex of the desired wedge to be removed. For example, the guide wire 8 may have a threaded end that engages the bone. Next, the first saw guide portion 27 and the second saw guide portion 34 are rotated to the desired wedge angle and the set screw 35 is tightened against the second cylindrical section 23 of the first saw guide to discourage relative movement between the first and second guides 20, 30.

After setting the angle, the apparatus 10 is threaded onto the guide wire 8 as generally shown in FIG. 3. Apparatus 10 is then rotated about guide wire 8 to the desired position and secured to the guide wire using set screw 25. At this point, the surgeon is ready to position the surgical saw between one of the two saw guides to make the necessary cuts. After the first cut is made, the surgeon continues to the other saw guide and makes the next cut.

Because the first saw guide portion and the second saw guide portion of apparatus 10 do not intersect, a small portion of the bone proximate the apex of the wedge will need to be cut without the aid of the apparatus 10. FIG. 9 illustrates the cuts made using an embodiment of the present invention. The distance between the ends of the cuts and the apex of the wedge is exaggerated for illustrative purposes. After removing the apparatus 10, the surgeon can use the cuts created using the apparatus 10 as a reference to guide the completion of the cuts. In other words, the surgeon can use the cut surfaces of the bone as a guide to extend the cuts to the apex.

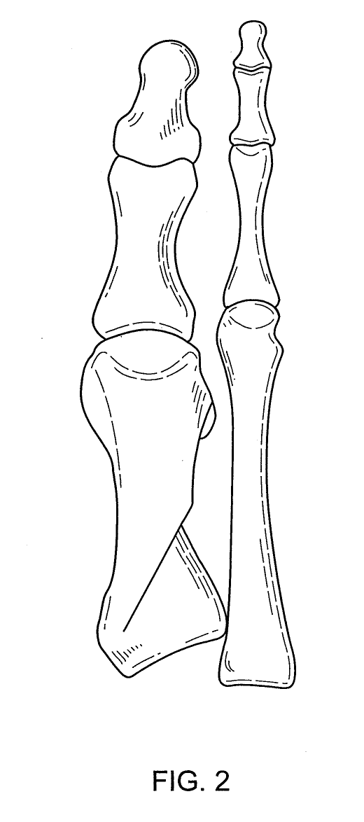

After all of the cuts are made and the wedge is removed, the bone may be reapproximated as generally shown in FIG. 2.

### ALTERNATIVE EMBODIMENTS

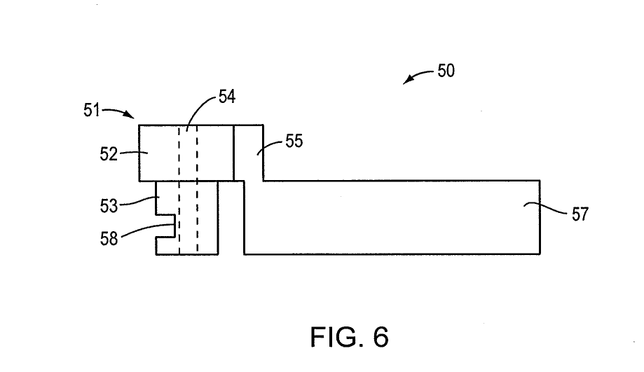

FIG. 6 illustrates an alternative embodiment of the first guide 50, which could be used in connection with the second guide 30. In this embodiment, the first guide 50 includes a base portion 51 having first and second cylindrical sections 52 and 53 respectively. The first cylindrical section 52 has a larger diameter than the second cylindrical section 53. A bore 54 is formed through the approximate center of the first and second cylindrical sections 52, 53. Extending from the first cylindrical section 52 is a saw guide support 55 that attaches the first diameter 52 to a saw guide portion 57. The saw guide portion 57 includes two substantially parallel walls spaced apart to accept a surgical saw as generally described with regard to first guide 20.

A notch 58 is formed along the length of the second cylindrical section 53 such that when assembled with the second guide 30 will generally align with the set screw 35. It should be understood that the notch 58 may extend to the free end of the second cylindrical section 53 as desired. This notch allows the set screw 35 to contact the guide wire 8 and urge it into contact with the inner surface of the bore 54. As a result, the set screw 35, when tightened, secures the first and second guides 50, 30 to the guide wire 8 thereby discouraging relative angular movement between the first and second guides 50, 30 and between the assembly and the guide wire 8. In this embodiment, only a single set screw is necessary.

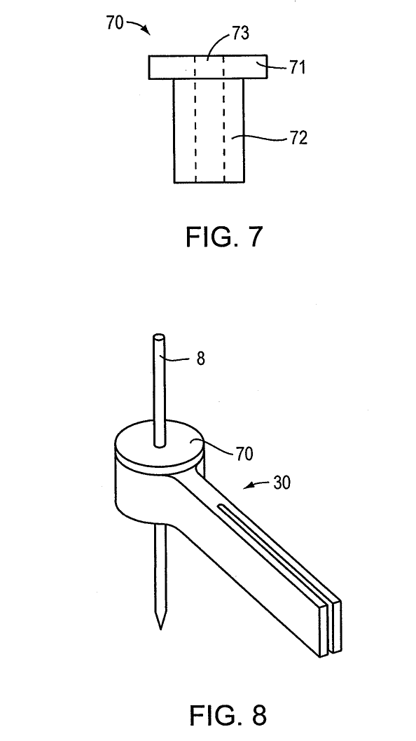

FIG. 7 illustrates a further embodiment of the present invention. In this embodiment, a spacer 70 in place of the first guide 20 and the second saw guide 30 would be used for linear cuts. The spacer 70 includes a first cylindrical section 71 and a second cylindrical section 72. A through bore 73 is formed along the central axis of the first and second cylindrical sections 71, 72. The purpose of this spacer is to facilitate connection of the second guide 30 to a guide wire 8 for use in linear osteotomy procedures.

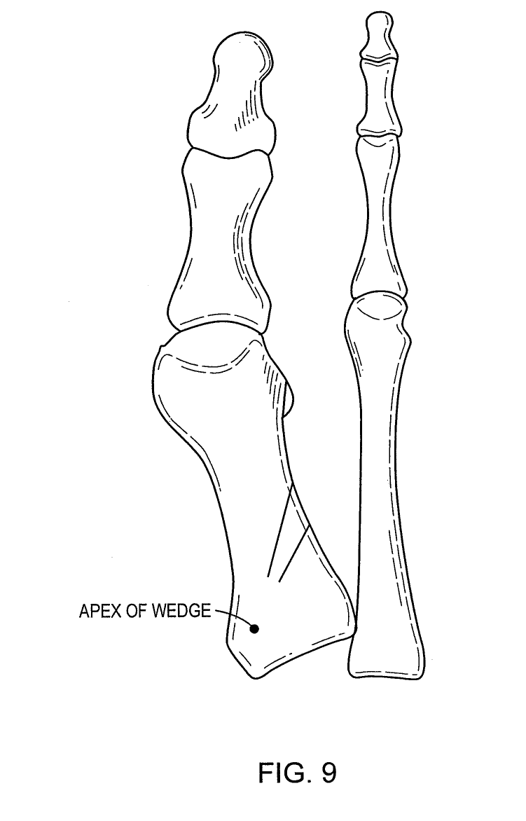

FIG. 8 illustrates the spacer 70 assembled to the second guide 30 in accordance with an embodiment of the present invention. As illustrated, this embodiment only includes a single saw guide portion. In one embodiment, the second guide 30 includes a threaded hole and set screw that engages the first cylindrical section 71 similar to that described with reference to apparatus 10 for discouraging movement with reference to the guide wire 8 when tightened. In another embodiment, second cylindrical section includes a notch as generally described with reference to first guide 50.

Angular osteotomy procedures that may benefit from the apparatuses and methods described in this application include:

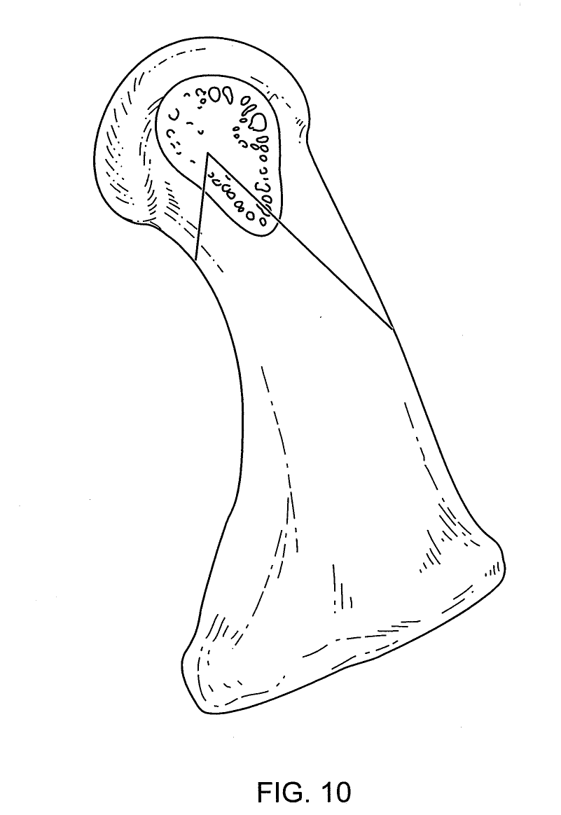

1. Austin or Chevron osteotomy of the first metatarsal, or modifications thereof such as the Kalish and Youngswick modification, for the correction of hallux valgus (bunion) deformity (See FIG. 10);

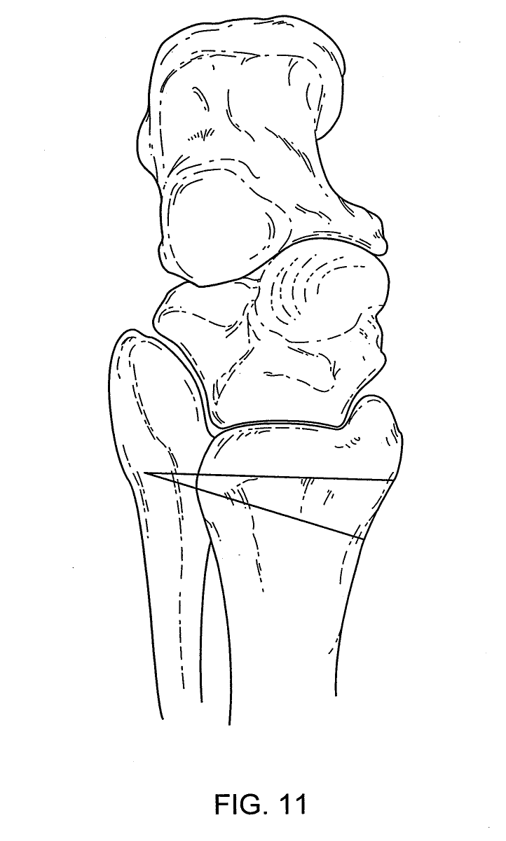

2. Supramallelolar osteotomy for correction of angular deformities involving the ankle joint (See FIG. 11);

3. High tibial osteotomy (HTO) for correction of congenital valgus or varus attitude of the tibia/leg, such as in congenital tibial varum (bow-legged) deformity, or for acute correction of malunion of the tibia after injury;

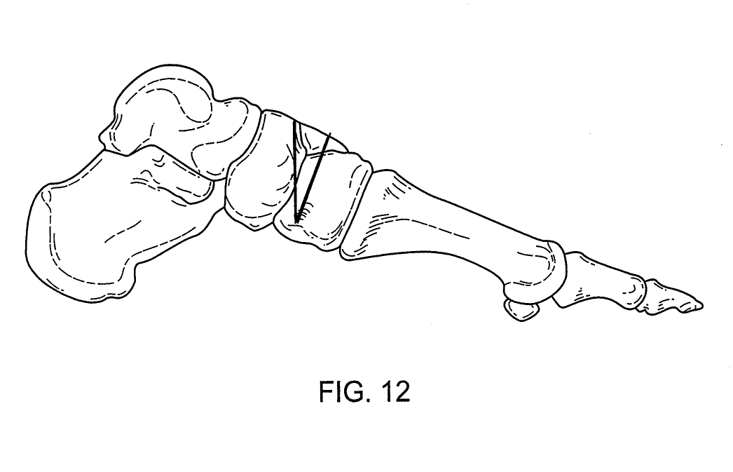

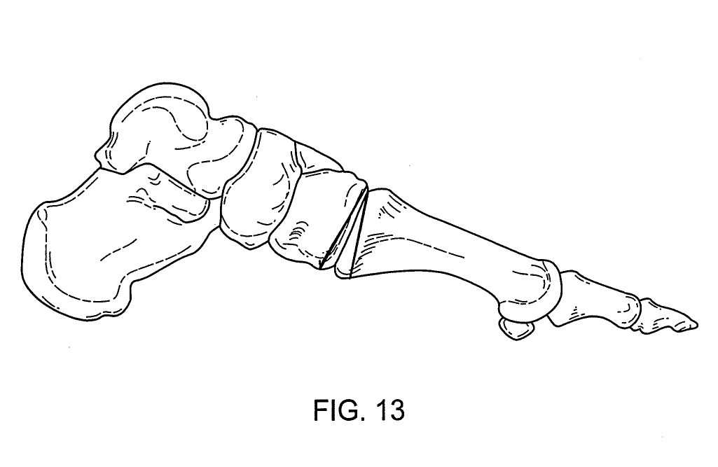

4. Midfoot angular osteotomies, such as a Cole or Japas-type osteotomy, designed to correct angular deformities of the foot associated with high and low longitudinal arch conditions (See FIG. 12); and

5. Fusion of a joint with acute angular correction. For example, removal of both sides of a joint for the purpose of reduction of the intermetatarsal angle and elimination of hypermobility of the first ray of the foot, as described by a Lapidus-type procedure (See FIG. 13).

Linear osteotomy procedures that may benefit from the apparatuses and methods described herein include:

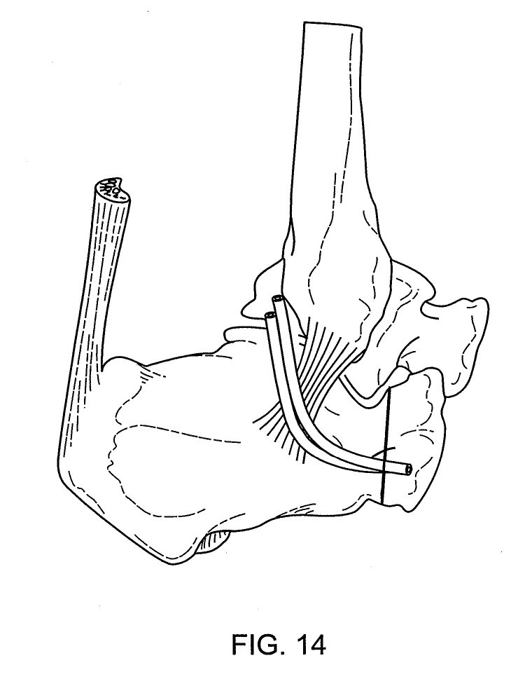

1. Evans-type osteotomy of the calcaneus for the correction of flatfoot deformity by means of placement of a bone graft for the purpose of lengthening the lateral column of the foot (See FIG. 14);

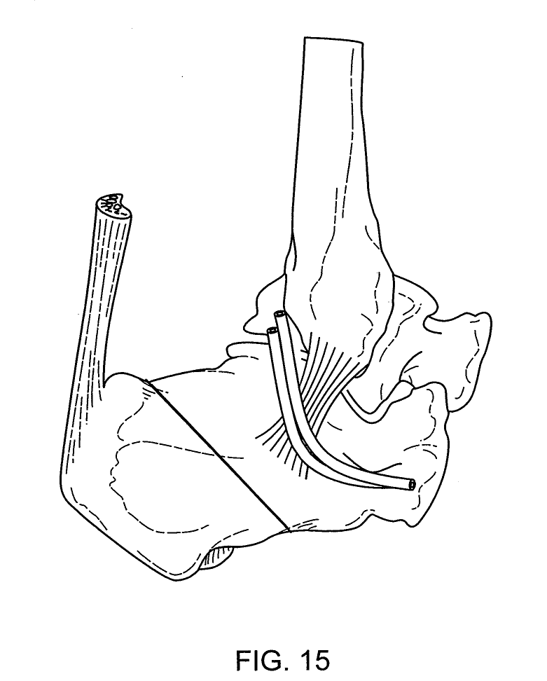

2. Dwyer-type osteotomy of the calcaneus, for the purpose of correction of varus and valgus alignment of the calcaneal tuber (See FIG. 15); and

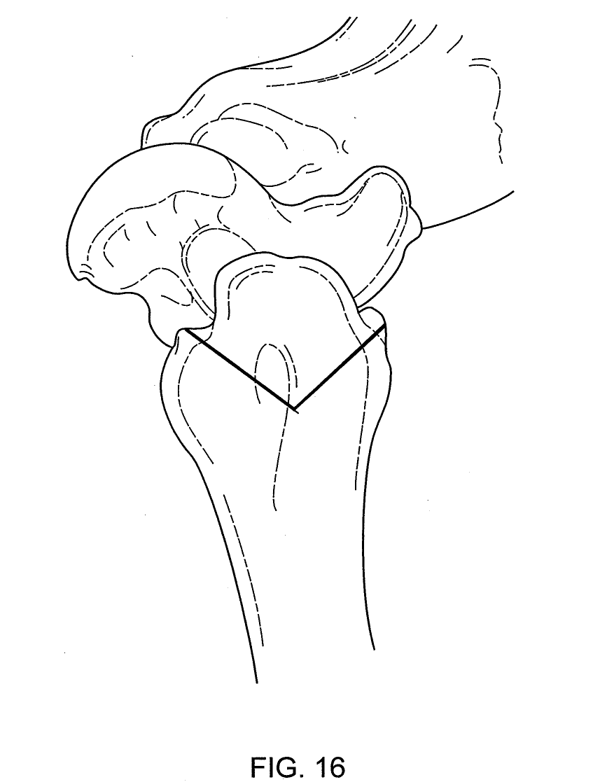

3. Medial malleoloar osteotomy for the purpose of exposure of the medial talar dome to remove and repair cartilage defects involving this portion of the ankle joint (See FIG. 16).

It should be understood, however, that the apparatuses and methods described herein may be used to facilitate other known or developed osteotomy procedures.

### CONCLUSION

Many modifications and other embodiments of the inventions set forth herein will come to mind to one skilled in the art to which these inventions pertain having the benefit of the teachings presented in the foregoing descriptions and the associated drawings. Although embodiments of the present invention were described in a medical context herein, one of skill in the art will appreciate that embodiments of the present inventions may be used in other contexts where angular cuts are made. For example, the embodiments of the present invention may be used in the general construction industry when making cuts in crown molding or stairway components. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. An apparatus for guiding a surgical saw during osteotomy procedures comprising:
a first saw guide having a first base portion with a first bore and a first guide portion comprising a pair of substantially parallel walls spaced apart to guide said surgical saw;
a second saw guide pivotably connected to said first saw guide, wherein said second saw guide includes a second base portion with a second bore and a second guide portion comprising a pair of substantially parallel walls spaced apart to guide said surgical saw; and
a locking mechanism configured to selectively discourage relative movement between said first saw guide and said second saw guide.

2. The apparatus of claim 1, wherein said first base portion includes a cylindrical section having substantially the same central axis as said first bore and sized to fit within and extend into said second bore.

3. The apparatus of claim 2, wherein said second base portion forms a threaded hole intersecting said bore adjacent a portion of said cylindrical section extending into said second bore and wherein said locking mechanism is a set screw configured to cooperate with said threaded hole and be selectively tighten against said cylindrical section.

4. The apparatus of claim 2 further comprising a guide wire having a first end configured to engage a bone and sized to cooperate with said first bore.

5. The apparatus of claim 4 further comprising a second locking mechanism configured to selectively discourage movement of said second saw guide relative to said guide wire.

6. The apparatus of claim 4, wherein said cylindrical section includes notch positioned to cooperate with said locking mechanism such that said locking mechanism can selectively engage said guide wire and discourage relative movement of said first saw guide and said second saw guide relative to said guide wire.

7. A method of aligning a surgical saw to make complementary cuts on a bone comprising the steps of:
securing a guide wire to a bone;
threading a first saw guide onto said guide wire wherein said first saw guide includes a pair of substantially parallel walls spaced apart to guide said saw;
threading a second saw guide onto said guide wire wherein said second saw guide includes a pair of substantially parallel walls spaced apart to guide said saw; and
selectively engaging a locking mechanism to discourage relative movement of said first saw guide relative to said guide wire.

8. The method of claim 7 further comprising the steps of:
selectively engaging a second locking mechanism to discourage relative movement of said first saw guide to said second saw guide.

9. The method of claim 8, wherein said step of selectively engaging a second locking mechanism occurs prior to the step of threading a first saw guide and the step of threading a second saw guide and the first and second saw guides are threaded onto said guide wire together.

10. The method of claim 7, further comprising the steps of:
making a first cut in said bone using said first saw guide;
making a second cut in said bone using said second saw guide;
removing said guide wire and said first and said second saw guides; and
extending said first cut using cut surfaces of said bone as a guide.

11. An apparatus for facilitating a linear cut in a bone comprising:
a saw guide having a base portion with a bore extending there through and a guide portion comprising a pair of substantially parallel walls spaced apart to guide a cutting apparatus;
a spacer having a second bore and a cylindrical section having substantially the same central axis as said second bore and sized to be received in said bore of said saw guide;
a locking mechanism configured to discourage relative movement between said saw guide and said spacer; and
a guide wire having a first end configured to engage a bone and sized to cooperate with said second bore.

12. The apparatus of claim 11, wherein said locking mechanism is a set screw configured to cooperate with a threaded hole formed in said saw guide and further configured to be selectively tightened against said cylindrical section.

13. The apparatus of claim 11 further comprising a second locking mechanism configured to selectively discourage movement of said saw guide relative to said guide wire.

14. The apparatus of claim 11, wherein said cylindrical section includes notch positioned to cooperate with said locking mechanism such that said locking mechanism can selectively engage said guide wire and discourage relative movement of said saw guide and said spacer in relation to said guide wire.

15. An apparatus for guiding a bone cutting apparatus comprising:
a first saw guide comprising (1) a first base portion forming a first bore and a cylindrical section having substantially the same central axis as said first bore and (2) a first guide portion comprising a pair of substantially parallel walls spaced apart to accept said cutting apparatus wherein said parallel walls of said first guide portion are substantially parallel with a longitudinal axis of said first bore;
a second saw guide comprising (1) a second base portion forming a second bore configured to receive said cylindrical section and forming a threaded hole intersecting with said second bore, and (2) a second guide portion comprising a pair of substantially parallel walls spaced apart to accept said cutting apparatus there between wherein said parallel walls of said second guide portion are substantially parallel with a longitudinal axis of said second bore; and
a locking mechanism comprising a set screw sized to cooperate with said threaded hole such that said set screw can be selectively tightened against said cylindrical section of said first saw guide to discourage relative movement of said first guide relative to said second guide.

16. The apparatus of claim 15, wherein said second base portion forms a second threaded hole which intersects said second bore and said apparatus further comprises:
a guide wire having a first end configured to engage a bone and sized to cooperate with said first bore; and
a second locking mechanism comprising a second set screw sized to cooperate with said second threaded hole such that said second set screw can be selectively tightened against said guide wire to discourage relative movement of said first and second saw guides in relation to said guide wire.
